(19) United States
(12) Patent Application Publication
(10) Pub. No.: US 2011/0245753 A1
(43) Pub. Date: Oct. 6, 2011

(54) APPARATUS AND METHOD FOR LOWERING INTRAOCULAR PRESSURE IN AN EYE

(76) Inventor: Murad A. Sunalp, Tulare, CA (US)

(21) Appl. No.: 13/080,530

(22) Filed: Apr. 5, 2011

Related U.S. Application Data
(60) Provisional application No. 61/320,960, filed on Apr. 5, 2010.

(51) Int. Cl. A61M 1/00

(52) U.S. Cl. 604/9

(57) ABSTRACT

An eye mounted device configured to actively evacuate fluid from the anterior chamber of the eye to an exterior region or the sclera of the eye. The device employs a miniature pump powered by a photovoltaic cell which is mounted in the clear corneal tissue and which may be curved in the same shape as the eye. One or a combination of pressure sensors and timers determine the duration the device evacuates fluid from the user's eye.

This application claims priority to U.S. Provisional Application No. 61/320,960 filed on Apr. 5, 2010, and is incorporated herein in its entirety.

### BACKGROUND OF THE INVENTION

1. Field of the Invention

The disclosed device relates to sclera and cornea implants. More particularly, it relates to a device implanted into the sclera or corneal tissue of the eye to provide a means to relieve excess intraocular pressure which frequently accompanies Glaucoma.

2. Prior Art

In the human eye, aqueous humor is a fluid secreted into the posterior chamber of the eye, by the ciliary body. From the secretion point, it flows through the narrow cleft between the front of the lens and the back of the iris, and then escapes through the pupil into the anterior chamber of the eye. From the anterior chamber, the fluid exits the eye via the trabecular meshwork. From the trabecular meshwork, the fluid exits into Schlemm's canal.

Aqueous humor is continually produced by the ciliary processes to maintain the shape of the eye and provide nourishment to various structures of the eye. As with any fluid entering a closed structure, the rate of production of the fluid must be balanced by an equal rate of aqueous humor drainage. Small variations in the production or outflow of aqueous humor will have a major influence on the intraocular pressure over time.

High intraocular pressure caused by a build up of aqueous fluid which accompanies Glaucoma is one of the main causes of damage to the optic nerve. Thus, the high pressure can be a major cause of sight loss in humans. The increase of pressure in some cases builds gradually, while in others may rapidly increase. For cases of a slow increase in pressure, drugs are conventionally employed as treatment and frequently work well. When a patient suffers from a rapid rise in such pressure or a frequent and long term rise in ocular pressure that can easily reach a dangerously high point, severe damage to the eye and permanent loss of sight can result.

A conventional treatment for intraocular pressure has been the use of surgery. One procedure known as anterior ciliary sclerotomy involves a series of incisions in the anterior sclera which has proved to lower intro ocular pressure. Unfortunately, following a period of time after the procedure the incisions heal and scar, causing the pressure to rise once again and risk the loss of sight in the patient.

Other surgical procedures and implantable devices have recently been developed to address the problem of intra ocular pressure build up.

U.S. Pat. No. 6,102,045 to Nordquist discloses a method and apparatus for lowering intraocular pressure by means of an implantable filtering member which extends into the anterior chamber of the eye through an opening in the limbus cornea. The device simply provides a passive means for drainage of fluid from the anterior chamber of the eye and by no means provides active evacuation of such fluid. Nordquist also lacks the provision of direct communication between the anterior chamber and the exterior region of the eye which may increase the risk of infection to the anterior chamber.

U.S. Pat. No. 5,178,604 to Baerveldt teaches the use of an implant for reducing pressure caused by Glaucoma by increasing eye drainage. However, Baerveldt is simply a passive tube which communicates directly between the interior chamber of the eye and an exit for the fluid, and offers only a passive means of drainage. Consequently, in patients with varying fluid pressure increases, or those with long term increases, the passive tube may lack the ability to communicate sufficient fluid volume to protect the patient.

In the field of active fluid communication, a pump is required. However, conventional pumps require batteries which must be changed, or may be recharged by induction if damage to the patient may be avoided.

As such, there is an unmet need, for an active means to drain the anterior chamber of the eye of fluid, to maintain intraocular pressure at a safe and healthy level. Such a device should be able to vary the rate of flow of fluid from the eye, to thereby maintain a healthy intraocular pressure which is neither too low or too high. Such a device should provide long term ability for pumping so as to avoid subsequent surgeries to replace batteries. Such a device should be programmable ideally to allow for future upgrades in performance based on new sensors and the like so as to provide a reliable means to actively evacuate fluid build up in the eye which is a major health threat to the patient when intraocular pressure passes beyond a safe point.

### SUMMARY OF THE INVENTION

The device herein provides a solution to the above noted shortcomings in the prior art. The device provides a means for active communication of fluid from the eye through a continuous evacuation of aqueous fluid from the anterior chamber of the eye to prevent harmful intraocular pressure.

The disclosed invention is composed of a pump sized to occupy a mounting site in the scleral tissue, such as the nanopump manufactured by Debiotech. However those skilled in the art will realize that many manufacturers make such extremely miniature devices and all considered within the scope of this intention.

The pump is engaged to a tubular conduit extending into the anterior chamber of the eye for fluid evacuation at a distal end, and the other to a conduit for fluid drainage outside the ocular chamber or the eye.

The pump is powered by a photovoltaic cell in a novel manner to provide energy to the pump. The photovoltaic cell is of sufficient size and dimension to generate electricity for the pump and concurrently allow for implantation into the cornea of the eye where the clear tissue allows for light transmission which is required for power generation for the pump. A slow discharge capacitor or battery may also be included to provide power when the patient is asleep or no light is available. Also, a data processor to control the pump to adapt the flow of fluid from the eye to keep the intraocular pressure at a safe level as well as to control the pump depending on whether it is on battery power or photo cell power or both.

In accordance with the described components of the invention, the nanopump is implanted into the scleral tissue of the eye adjacent to the cornea. The photovoltaic cell is implanted into the cornea of the eye and is engaged to the nanopump by some means to allow for electrical communication of the generated power. The cornea, being transparent in nature, allows the photovoltaic cell to directly capture incoming sunlight and power the nanopump. The light may be sunlight or in the case of most photo cells, room light.

The intake conduit of the nanopump as described has a distal end positioned in communication with the anterior chamber of the eye to communicate aqueous fluid therefrom to prevent excess intraocular pressure in the eye. The outflow conduit extends from the pump to an outer region of the eye where the aqueous fluid is evacuated or communicated to blood vessels naturally. Similarly, the outflow conduit may be inserted into the sclera of the eye itself where fluid can be absorbed or diffused to the outer region of the eye.

In a preferred mode of the invention, the nanopump may be activated by means of a pressure sensor in communication with a portion of the eye having intraocular pressure. The pressure sensor may be, as such, employed within the anterior chamber of the eye and when it reaches a predetermined pressure, the pump is activated and the excess aqueous fluid is evacuated. Other means of activation of the device may be predetermined time intervals or whenever the photovoltaic cell has reached a predetermined energy level.

With respect to the above description, it is to be understood that the invention is not limited in its application to the details of operation of the device nor the arrangement of the components or steps in the method set forth above or in the following descriptions or in the illustrations in the drawings. The various methods of implementation and operation of the disclosed device herein, are capable of other embodiments and of being practiced and carried out in various ways which will be obvious to those skilled in the art once they review this disclosure. Also, it is to be understood that the phraseology and terminology employed herein are for the purpose of description and should not be regarded as limiting.

Therefor, those skilled in the art will appreciate that the conception upon which this disclosure is based may readily be utilized as a basis for designing similar devices for carrying out the several purposes of the present invention. Therefor, the objects and claims herein should be regarded as including such equivalent constructions, steps, and methodology insofar as they do not depart from the spirit and scope of the present invention.

It is an object of this invention to provide a means to actively evacuate aqueous fluid build up within the anterior chamber of the eye by means of a nano scale pump.

It is another object of this invention to be sufficiently powered for long term use by means of intra-corneal insertion of a photovoltaic cell.

Yet another object of the device is to employ a pressure sensitive activation switch for the pump.

A still further object is the provision of a means to store energy from the photocell as a reservoir to be employed when needed.

These and further objectives of this invention will be brought out in the following part of the specification.

### BRIEF DESCRIPTION OF THE FIGURES

FIG. 1 is an depiction of a preferred embodiment of the device with simplified nanopump and photovoltaic cell, the pressure sensor and microprocessor.

FIG. 2 shows an eye ball depicting an incision as a possible point of insertion of the device near the cornea.

FIG. 3 shows the device and its placement in the scleral layer of the eye.

FIG. 4 depicts a preferred mode of the device in the as used position within the scleral and corneal layers of the eye.

### DETAILED DESCRIPTION OF THE PREFERRED EMBODIMENTS OF THE DEVICE

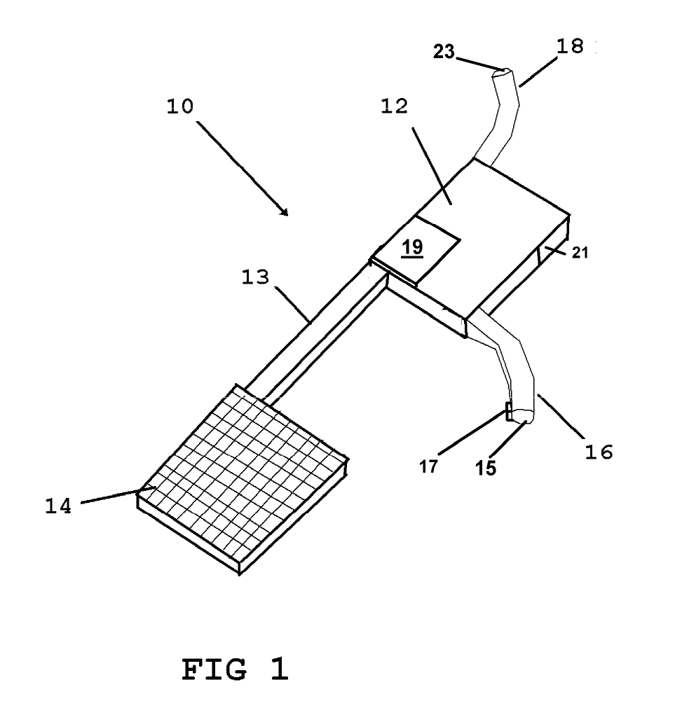

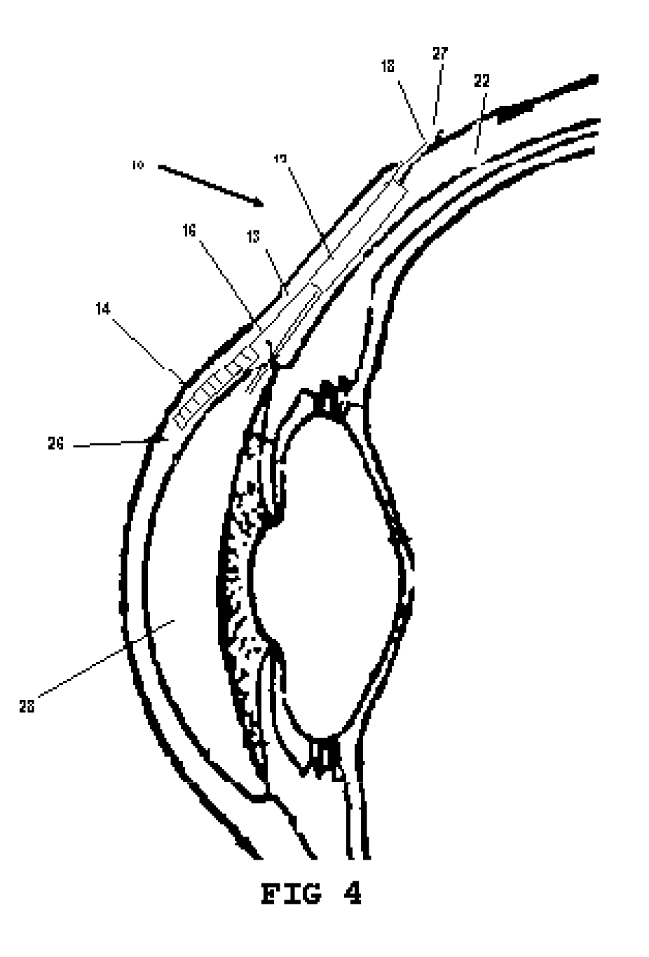

Now referring to drawings in FIGS. 1-4, wherein similar components are identified by like reference numerals, there is seen in FIG. 1 a preferred embodiment of the device 10. As shown, a nanopump 12 is positioned in an operative mounted position in the eye tissue and in an electrical connection 13 to the photovoltaic cell 14 which is operatively mounted within the clear tissue of the sclera to provide maximum light transmission thereto from outside the eye. The orientation, location, and relative size of the components of the device 10 are not limited to the depictions set forth in the figure but merely give simplified versions of the components for demonstration purposes and should not be considered limited as those skilled in the art will realize that nano-sized pumps and photovoltaic cells are made by many manufacturers all of which are considered within the scope of this intention.

A fluid intake conduit 16 having an intake aperture 15 on its distal end communicates fluid to the nanopump 12. An outflow conduit 18 is connected to the nanopump 12 at a first end and has a relative size and dimension to adequately transport fluid from the nanopump 12 to an exit aperture 23 at a flow rate determined output by the nanopump 12.

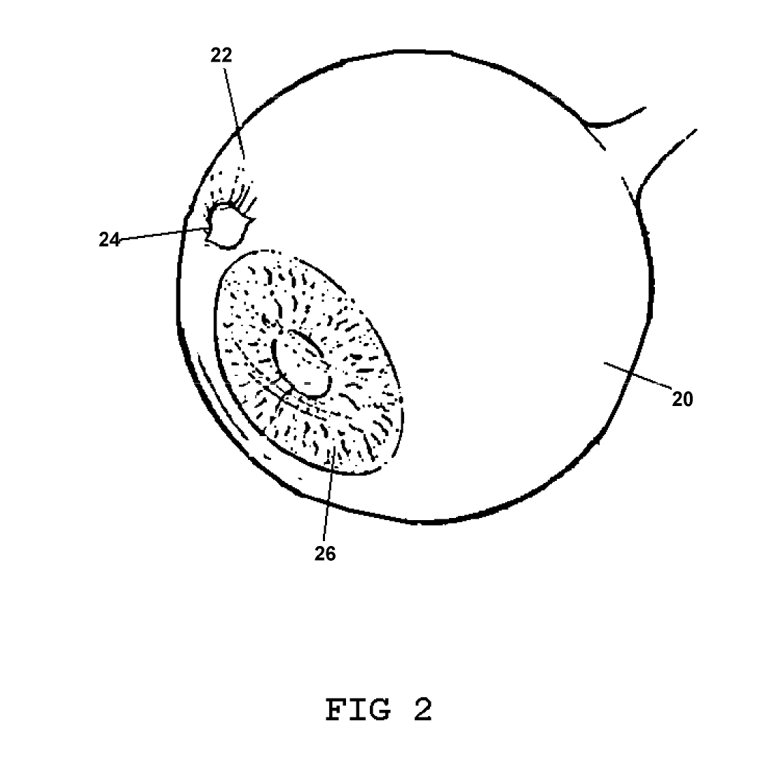

FIG. 2 shows one typical incision 24 to be made to the sclera 22 of the eye 20 adjacent to the cornea 26 for insertion of the device 10. The method for insertion of the device 10 requires incisions to be made in the sclera 22 to allow for the insertion of the photovoltaic cell 14 into a pocket formed by the incision and allow communication of power from the photovoltaic cell 14 located in the cornea 26.

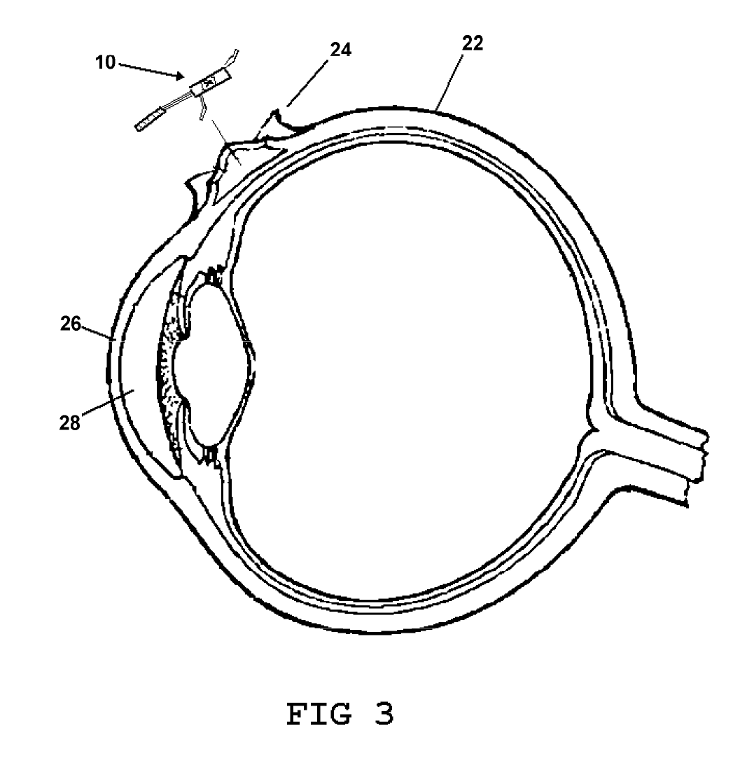

FIG. 3 shows a cut side view of the eye 20 where the device 10 is oriented with the photovoltaic cell 14 positioned in a pocket or behind the clear tissue of the cornea 26 of the eye. The photovoltaic cell 14 may preferably be curved to allow it to follow the curve of the eye and maximize light reception. Similarly, the outflow conduit 18 is oriented to evacuate fluid to an exit aperture 23 at or near the exterior region of the eye or into the webbed area in or adjacent to the sclera 22 for absorption by blood vessels.

Once the nanopump 12 is implanted to a mounted position in the eye, such as a formed pocket in the sclera, the photovoltaic cell 14 which is inserted into a mount in a pocket within the cornea 26 or behind the cornea 26 may be electrically engaged to the device 10. Electrical connection is maintained between the nanopump 12 and photovoltaic cell 14 by means of wires 13. Incoming light to the cornea 26 will pass through to the cornea 25 to the surface of the photovoltaic cell 14 and be communicated by the wire 13 to power the device 10.

Upon insertion, the intake aperture 15 at the distal end of the intake conduit 16 is placed in a fluid communication with the anterior chamber 28 where aqueous fluid builds pressure during a rising of intraocular pressure. A means to activate the device 10 may be provided by a pressure sensing device 17 to act as a switch for the wire 13 or in communication with a microprocessor 19 having software adapted to the task and located on or near the nanopump 12 to cause the pump to activate. The pressure sensing device 17 would be in electrical communication with the device 10 using wires 13 or other means. The pressure sensing device 17 located at an interior eye pressure activation point as determined by a physician, would cause the pump 12 to begin pumping once a certain pressure is reached within the anterior chamber 28 or another interior portion of the eye 20. The device 10 is powered ‘on’ with the nanopump 12 causing an evacuation of fluid until a desired lower pressure level is sensed as obtained by the pressure sensing device 17. The exit aperture 23 at the distal end of the outflow conduit 18 which is in fluid communication with the outflow side of the nanopump 12, can be positioned toward the exterior region 27 of the eye 20 or within the sclera 22 for absorption or diffusion to the exterior.

The activation of the device 10 to trigger the nanopump 12 to run and evacuate fluid may also be based on regularly scheduled timing points and for durations of time as determined by a physician and activated by a microprocessor 19. Or the device 10 can employ both timing and durations and sensors to activate a pumping by the device. Power may be stored onboard the device 10 in a electrical power storage means 21 such as a battery or slow discharge capacitor. This allows the device 10 to function when no light or little light is available for electrical power generation by the photovoltaic cell 14 such as when the patient is sleeping or in a darkened location.

While all of the fundamental characteristics and features of the invention have been shown and described herein, with reference to particular embodiments thereof, a latitude of modification, various changes and substitutions are intended in the foregoing disclosure and it will be apparent that in some instances, some features of the invention may be employed without a corresponding use of other features without departing from the scope of the invention as set forth. It should also be understood that various substitutions, modifications, and variations may be made by those skilled in the art without departing from the spirit or scope of the invention. Consequently, all such modifications and variations and substitutions are included within the scope of the invention as defined by the following claims.

What is claimed is:

1. An apparatus configured for implantation in the eye to relieve intraocular pressure, comprising:
a pump sized for implant into a cavity formed in the scleral tissue of the eye of a patient;
an intake conduit extending from said pump to an intake aperture at a distal end;
said intake conduit having a length providing a means to position said intake aperture in a communication with the anterior chamber of said eye;
an outflow conduit extending from a first end in communication with said pump, to an outflow aperture at a distal end of said outflow conduit;
said outflow conduit having a length configured to position said outflow conduit within a layer of said scleral tissue or exterior to scleral tissue and said eye;
a photovoltaic cell in an electrical communication with said pump, said photovoltaic cell configured for a mounting within the cornea of said eye;
said photovoltaic cell communicating electric energy to power said pump to an energized state using a conversion of light communicated thereto through said cornea;
said pump in said energized state providing a pumping of fluid from said anterior chamber to an exit at said outflow aperture; and
said pumping of fluid providing a means to lower and maintain an intraocular pressure of said eye.

2. The apparatus configured for implantation in the eye of claim 1, additionally comprising:
pressure sensing means to switch said pump to and from said energized state; and
said pressure sensing means switching said pump to said energized state only when sensed intraocular pressure rises to a preset level, whereby said pump is activated for said pumping of said fluid only when said intraocular pressure dictates a need for it.

3. The apparatus configured for implantation in the eye of claim 1, additionally comprising:
means for storage of electrical energy for subsequent communication to said pump; and
said means for storage of electrical energy in said electrical communication with said photovoltaic cell, whereby a portion of said electric energy from said photovoltaic cell can be stored for a said subsequent communication to said pump.

4. The apparatus configured for implantation in the eye of claim 2, additionally comprising:
means for storage of electrical energy for subsequent communication to said pump; and
said means for storage of electrical energy in said electrical communication with said photovoltaic cell, whereby a portion of said electric energy from said photovoltaic cell can be stored for a said subsequent communication to said pump.

5. The apparatus configured for implantation in the eye of claim 1, additionally comprising:
a microprocessor having software adapted to switching said electrical energy in a communication in-between said electric energy and said pump; and
said microprocessor employing said software to place said pump in said energized state for time durations.

6. The apparatus configured for implantation in the eye of claim 2, additionally comprising:
a microprocessor having software adapted to switching said electrical energy in a communication in-between said electric energy and said pump; and
said microprocessor employing said software to place said pump in said energized state for one or a combination of intermittent time durations or pumping durations subsequent to when a sensed intraocular pressure rises to a preset level.

7. The apparatus configured for implantation in the eye of claim 3, additionally comprising:
a microprocessor having software adapted to switching said electrical energy in a communication in-between said electric energy and said pump; and
said microprocessor employing said software to place said pump in said energized state for one or a combination of intermittent time durations or pumping durations subsequent to when a sensed intraocular pressure rises to a preset level.

8. The apparatus configured for implantation in the eye of claim 4, additionally comprising:
a microprocessor having software adapted to switching said electrical energy in a communication in-between said electric energy and said pump; and
said microprocessor employing said software to place said pump in said energized state for one or a combination of intermittent time durations or pumping durations subsequent to when a sensed intraocular pressure rises to a preset level.

9. A method of employing the apparatus of claim 1 for lowering the intraocular pressure of a patient comprising the steps of:
forming said cavity in the scleral tissue of a patient and positioning said pump therein;
positioning said intake conduit to extend from said pump to position said intake aperture in a communication with the anterior chamber of said eye;
positioning said outflow conduit to extend from said pump and position said outflow aperture within a layer of said scleral tissue or at a position exterior to scleral tissue at the exterior surface of said eye;
positioning said photovoltaic cell within the cornea of said eye;
allowing said pump to reach said energized state to thereby provide said pumping of said fluid from said anterior chamber to an said outflow aperture and provide said means to lower and maintain an intraocular pressure of said eye.

10. The method of claim 9 comprising the additional steps of:
positioning a pressure sensor in an operative communication with said anterior chamber; and
employing said pressure sensor as a switch to move said pump to said energized state when said intraocular pressure surpasses a preset level.
